SEQUENCE LISTING (1 sequence)

SEQ ID NO: 1 - protein, 322 aa, Enterococcus faecium
MSYYFEIRIILPEEENQFLNRKLSKSELSEVTHYLQQKTSRGIPVKFRVGIFRVEDQTKIMSVTLNTKNTKETDVINLLLNRVTDQHVLVYLNEPTEPTLNTQELNRQELKTSNERQEIPQTEIPTETVNEPSVIKKISKKNQAKQTNSRKESLSESITKKNVPKIHLFISILTLFIVLLIGISVIQQVQLQSVKKESELLEEQIERVKETDISQSKIDTFGRYFLTYYFSQEKNQENYQSSLRTYVSEKVDISDWKALGKTLKSVNYYGSEQTKKGYSVEYLLNVSVDNRSKMQKITFEVEPTKNGFLVTTQPKLTDFSFN

(19) United States
(12) Patent Application Publication
(10) Pub. No.: US 2013/0309264 A1
(43) Pub. Date: Nov. 21, 2013

(54) NOVEL ANTIGEN OF ENTEROCOCCAL PATHOGENS AND USE THEROF AS VACCINE COMPONENT FOR THERAPY AND/OR PROPHYLAXIS

(71) Applicants: Johannes Hübner, Freiburg (DE); Elisabeth Grohmann, Berlin (DE); Andrea Kropec-Hübner, Freiburg (DE)

(72) Inventors: Johannes Hübner, Freiburg (DE); Elisabeth Grohmann, Berlin (DE); Andrea Kropec-Hübner, Freiburg (DE)

(73) Assignee: UNIVERSITÄTSKLINIKUM FREIBURG, FREIBURG (DE)

(21) Appl. No.: 13/879,750

(22) PCT Filed: Nov. 3, 2011
(86) PCT No.: PCT/EP11/69363

§ 371 Date: Aug. 5, 2013

(30) Foreign Application Priority Data
Nov. 5, 2010 (EP) 10190167.6

(51) Int. Cl. C07K 14/24

(52) U.S. Cl. CPC C07K 14/24
USPC 424/190.1; 530/350; 435/7.1

(57) ABSTRACT

The present invention relates to antigens, more particularly protein antigens of enterococcal pathogens which are useful as vaccine components for therapy and/or prophylaxis.

The present invention relates to antigens, more particularly protein antigens of enterococcal pathogens which are useful as vaccine components for therapy and/or prophylaxis.

### BACKGROUND OF THE INVENTION

Enterococci are among the most important pathogens associated with infections in hospitalized patients. Especially the presence of multiple antibiotic resistance determinants in most clinically relevant isolates urges the development of alternative treatment and prevention strategies to combat these sometimes untreatable infections. Enterococci have developed specific mechanisms to acquire and transmit DNA horizontally, and these traits are responsible for numerous outbreaks in the hospital setting.

Bacterial conjugation is the most important means of gene delivery enabling adaptation of bacteria to changing environmental conditions including spread of antibiotic resistance genes, thereby generating multiply antibiotic resistant pathogens. Multiply resistant pathogens, such as Enterococcus faecalis, Enterococcus faecium, Pseudomonas aeruginosa, Streptococcus pneumoniae, and Staphylococcus aureus represent a serious threat to antibiotic treatment of hospitalized and immuno-suppressed patients (Grohmann 2006).

Bacterial conjugation systems are specialized types of type IV secretion systems (T4SS) dedicated to transport proteins (e.g., virulence factors, toxins) from bacterial pathogens to mammalian hosts. The conjugative T4SS have evolved to intercellularly transport DNA substrates in addition to proteins (Grohmann et al., 2003, Grohmann 2006).

pIP501 is a broad-host-range conjugative plasmid, originally isolated from the human pathogen Streptococcus agalactiae (Schaberg et al., 1982), the causative agent of sepsis and meningitis. It shows the broadest host range of conjugative plasmid transfer of any known mobile genetic element from Gram-positive bacteria, comprising virtually all studied Gram-positive bacteria including even filamentous multicellular Streptomyces and Gram-negative Escherichia coli (Kurenbach et al., 2003). Plasmid transfer requires a 15-kb plasmid-encoded transfer region comprising a complete T4SS encoding 15 transfer proteins. Several of these proteins have been characterized in some enzymatic detail, such as the T4SS ATPases OrfS and Orf10 which deliver energy for the DNA transport process by the hydrolysis of ATP.

The Orf7 protein was demonstrated to be a lytic transglycosylase with cleavage activity on peptidoglycan isolated from Gram-positive E. faecalis, as well as from Gram-negative E. coli.

A complete protein-protein interaction map has been established for the 15 T4SS proteins by yeast two-hybrid assay and pull-down. On the basis of these data, the first molecular model for a T4SS from Gram-positive pathogens has been developed (Abajy et al., 2007).

The T4SS proteins can be divided into three families with respect to their function in the transfer process: the energy delivery family, the channel component family, and the putative surface proteins or adhesins (Alvarez-Martinez and Christie, 2009). Orf5 and Orf10 are members of the energy component family, Orf15 is a putative member of the surface protein family, and the lytic transglycosylase Orf7 and Orf13 belong to the channel component family.

For Orf13, a scaffold function for the portion of the T4SS channel extending distally from the membrane, as shown for the T4SS subunits VirB8 and VirB10 of the Agrobacterium tumefaciens T-DNA transfer system, has been proposed (Alvarez-Martinez and Christie, 2009). Orf13 has one predicted transmembrane helix (Abajy 2007). In E. faecalis JH2-2 cell fractions Orf13 was localized to the cell envelope using immunoblot with anti-Orf13 antibodies. No protein-protein interactions with other T4SS proteins could be identified.

US 2005-026218 describes isolated nucleic acids that encode polypeptides that interact with T4SS, in particular the Rickettsia sibirica rsib_orf.1266 polypeptide.

Since infections caused by enterococci are of globally increasing importance, considerable effort has been devoted to the development of new treatment strategies against these pathogens. The main protective defense mechanism of the human immune system against enterococci is phagocytosis, which may occur through direct recognition of certain enterococcal surface structures or through opsonization by antibodies and complement.

The opsonophagocytic assay has been used to simulate the immune response in vitro and to identify enterococcal virulence factors. However, only few antigens have been identified so far that may offer the potential of inducing a protective immune response, and therefore would be promising vaccine targets.

It is therefore an object of the present invention to provide a new promising vaccine target for an active or passive immunotherapy of bacteria, and in particular enterococci. It is another object of the present invention, to provide novel and effective vaccines based on said target.

According to a first aspect thereof, these objects have been solved by the present invention by providing an isolated peptide selected from a) a protein comprising an Orf13 sequence according to SEQ ID No. 1, b) a homolog of Orf13 being to at least 80% identical with said protein on the amino acid level, c) an immunogenic peptide derived from said protein according to a) or b), and d) an antibody or antigenic fragment thereof that specifically binds to any of a) to c) for use in the treatment of diseases.

Orf13 as isolated from the Enterococcus faecalis plasmid pIP501 has the following amino acid sequence according the GenBank Accession number AJ505823.1:

(SEQ ID No. 1)
MSYYFEIRIILPEEENQFLNRKLSKSELSEVTHYLQQKTSRGIPVKFRVGIFRVEDQTKI
MSVTLNTKNTKETDVINLLLNRVTDQHVLVYLNEPTEPTLNTQELNRQELKTSNERQ
EIPQTEIPTETVNEPSVIKKISKKNQAKQTNSRKESLSESITKKNVPKIHLFISILTLFIVLL
IGISVIQQVQLQSVKKESELLEEQIERVKETDISQSKIDTFGRYFLTYYFSQEKNQENYQ
SSLRTYVSEKVDISDWKALGKTLKSVNYYGSEQTKKGYSVEYLLNVSVDNRSKMQK
ITFEVEPTKNGFLVTTQPKLTDFSFN

In addition, Orf13-homologs have been identified in two E. faecium strains E1679 (catheter tip isolate) and on the E. facium plasmid pVEF3 (protein p51); on five E. faecalis strains: HIP11704 (unnamed plasmid), E. faecalis DS5 (unnamed plasmid), E. faecalis RE25 (plasmid pRE25, T4SS almost identical with pIP501), E. faecalis DS5 (plasmid pAMbetal), and E. faecalis ADM24818, and in one S. pyogenes strain (plasmid pSM19035) as follows:

| Bacterial | | Acc. No. (as of |
|---|---|---|
| strain | Isolate/note/ref. | Nov. 1, 2010) |
| E. faecium | strain E1679; catheter tip | ZP_06698913.1 |
| | isolate | |
| E. faecium | plasmid pVEF3; protein p51 | YP_001974820.1 |
| E. faecalis | HIP11704 | ZP_05569726.1 |
| E. faecalis | DS5 | ZP_05563503.1 |
| E. faecalis | RE25; pRE25, T4SS almost | YP_783921.1 |
| | identical with pIP501 | |
| E. faecalis | DS5; plasmid pAMbeta1 | YP_003305375.1 |
| E. faecalis | ADM24818; Zhu et al., 2010 | ADM24818.1 |
| S. pyogenes | plasmid pSM19035 | YP_232773.1 |

Therefore, ORF13 seems to be present in several pathogenic bacteria, such as, for example, E. faecalis and E. faecium strains, and thus is a promising vaccine target for active or passive immunotherapy of, for example, enterococci.

Since appropriate animal models for an effective analysis of the immunogenicity and protective efficacy towards bacterial virulence factors are missing, the opsonophagocytic assay is used as a surrogate to simulate the mammalian in vivo immune response in vitro, and to identify enterococcal virulence factors that can be developed into, for example, effective antibacterial vaccines (Markus Hufnagel, Steffi Koch, Andrea Kropec, Johannes Huebner: Opsonophagocytic assay as a potentially useful tool for assessing safety of enterococcal preparations. International Journal of Food Microbiology 88 (2003) 263-267).

For Orf13, a scaffold function for the portion of the T4SS channel extending distally from the membrane, as shown for the T4SS subunits VirB8 and VirB10 of the Agrobacterium tumefaciens T-DNA transfer system, has been proposed (Alvarez-Martinez and Christie, 2009). Orf13 has one predicted transmembrane helix (Abajy 2007). In E. faecalis JH2-2 cell fractions Orf13 was localized to the cell envelope using immunoblot analysis with anti-Orf13 antibodies. Therefore, preferred is the peptide according to the present invention, wherein said peptide is a channel-forming component, and preferably has the activity to build portion of the T4SS channel.

Another aspect of the present invention then relates to an antibody or antigenic fragment thereof that specifically binds to any of the Orf13 peptides as used in the treatment of diseases in the context of the present invention. Preferred is a peptide according to the present invention, wherein said antibody or antigenic fragment thereof is selected from a monoclonal antibody, an scFV fragment and a Fab-fragment. The antibodies can further be human or humanized antibodies or fragments. Methods to produce antibodies are known to the person of skill and described in the respective literature.

The present invention provides new therapeutic approaches in order to treat bacterial infections, such as infections caused by Enterococcus faecalis, Enterococcus faecium, Pseudomonas aeruginosa, Streptococcus pneumoniae, Staphylococcus aureus and coagulase-negative staphylococci, as well as strains of Clostridia, Listeria or S. pyogenes, and in particular bacterial infections that are caused by strains showing resistance against multiple antibiotics, such as multiple resistant Enterococcus faecalis, Enterococcus faecium, Pseudomonas aeruginosa, Streptococcus pneumoniae, Staphylococcus aureus and coagulase-negative staphylococci, as well as strains of Clostridia or Listeria. Respective multiply resistant strains are also described in the literature. It is therefore a further aspect of the present invention to particular provide a treatment of such strains. In this context, it is particular preferred that said homolog of Orf13 is encoded by a plasmid isolated from such an antibiotic resistant strain of E. faecium, E. faecalis, or S. pneumonia.

Another aspect of the present invention then relates to a pharmaceutical composition comprising the peptide according to the present invention (i.e. at least one of a protein comprising an Orf13 sequence according to SEQ ID No. 1, a homolog of Orf13 being to at least 80% identical with said protein on the amino acid level, an immunogenic peptide derived from said protein according to SEQ ID No. 1 or said homolog, or an antibody or antigenic fragment thereof that specifically binds to any of SEQ ID No. 1 or said homolog), and a pharmaceutically acceptable carrier, adjuvant and/or diluent. Preferably, said pharmaceutical composition is effective for the prevention of a bacterial, and particularly an enterococcal infection.

Preferably, the pharmaceutical composition according to the present invention is a vaccine. Thus, according to the present invention to another aspect, there are provided vaccine compositions comprising one or more peptides according to the present invention (i.e. at least one of a protein comprising an Orf13 sequence according to SEQ ID No. 1, a homolog of Orf13 being to at least 80% identical with said protein on the amino acid level, an immunogenic peptide derived from said protein according to SEQ ID No. 1 or said homolog, or an antibody or antigenic fragment thereof that specifically binds to any of SEQ ID No. 1 or said homolog) in admixture with a pharmaceutically acceptable carrier diluent or adjuvant. Pharmaceutically acceptable carriers also include tetanus toxoid or diphteria toxoid. The pharmaceutical composition according to the invention can further comprise at least one cytokine.

Suitable adjuvants include oils i.e. Freund's complete or incomplete adjuvant; salts i.e. AlK(SO₄)₂, AlNa(SO₄)₂, AlNH₄(SO₄)₂, silica, kaolin, carbon polynucleotides, i.e. poly IC and poly AU. Preferred adjuvants include QuilA and Alhydrogel.

Another aspect of the present invention then relates to a pharmaceutical composition, wherein the peptide is covalently bonded or conjugated to an immunocarrier, such as capsular polysaccharides.

Vaccines of the invention may be administered parenterally by injection, rapid infusion, nasopharyngeal absorption, dermoabsorption, or buccal or oral. Said vaccine can also be formulated for administration via intramuscular, subcutaneous, or inhalation routes.

Another aspect of the present invention then relates to the peptide or the pharmaceutical composition according to the present invention for the prophylactic or therapeutic treatment of a disease or condition caused by a bacterium, and/or diseases and symptoms mediated by bacterial infections, such as, for example, enterococcal infection, urinary tract infections, bacteremia, bacterial endocarditis, peritonitis, wound or soft-tissue infections, pneumonia, and meningitis. Preferably, said bacterium is selected from enterococci, staphylococci or streptococci, such as, for example, E. faecium, E. faecalis, S. aureus or S. pyogenes, and in particular antibiotic-resistant strains thereof, and further preferably strains showing resistance against multiple antibiotics, such as multiple resistant Enterococcus faecalis, Enterococcus faecium, Pseudomonas aeruginosa, Streptococcus pneumoniae, and Staphylococcus aureus. Another aspect of the present invention then relates to use of the peptide or the pharmaceutical composition according to the present invention for the prophylactic or therapeutic treatment of a disease or condition as described here.

In a particularly preferred embodiment, the vaccines are administered to those individuals at risk of bacterial, and in particular enterococcal infection, such as infants, elderly and immunocompromised individuals.

As used in the present application, the term “individuals” include mammals. In a further embodiment, the mammal is human.

Vaccine compositions are preferably administered in a unit dosage form of about 0.001 to 100 Pg/kg (Peptide/body weight) and more preferably 0.01 to 10 Pg/kg and most preferably 0.1 to 1 Pg/kg 1 to 3 times with an interval of about 1 to 6 week intervals between immunizations.

Another aspect of the present invention then relates to a method for producing a pharmaceutical composition according to the present invention, preferably a vaccine, comprising admixing at least one of a protein comprising an Orf13 sequence according to SEQ ID No. 1, a homolog of Orf13 being to at least 80% identical with said protein on the amino acid level, an immunogenic peptide derived from said protein according to SEQ ID No. 1 or said homolog, or an antibody or antigenic fragment thereof that specifically binds to any of SEQ ID No. 1 or said homolog with a pharmaceutically acceptable carrier diluent or adjuvant as described above.

Another aspect of the present invention then relates to a method for therapeutic or prophylactic treatment of a disease or condition caused by a bacterium, and/or diseases and symptoms mediated by bacterial infections, such as, for example, bacterial infection, enterococcal infection, urinary tract infections, bacteremia, bacterial endocarditis, peritonitis, wound and soft tissue infections, and meningitis, or pneumonia in an individual, comprising administering to said individual a therapeutic or prophylactic amount of a pharmaceutical composition according to the present invention, preferably a vaccine. Preferably, said disease or condition is caused by a bacterium selected from enterococci, staphylococci or streptococci, such as, for example, E. faecium, E. faecalis, S. aureus or S. pyogenes, and in particular antibiotic-resistant strains thereof, and further preferably strains showing resistance against multiple antibiotics, such as multiple resistant Enterococcus faecalis, Enterococcus faecium, Pseudomonas aeruginosa, Streptococcus pneumoniae, and Staphylococcus aureus or coagulase-negative staphylococci.

Another aspect of the present invention then relates to a method for inducing an immune response against bacterial infection and in particular enterococcal infection, in a vertebrate, comprising administering to said vertebrate an immunologically effective amount of the pharmaceutical composition, and preferably a vaccine, according to the present invention.

Preferred is a method for the treatment and/or prophylaxis as described herein, wherein said method comprises administering intramuscularly, subcutaneously, or via inhalation to said vertebrate a therapeutically effective amount of a pharmaceutical composition, and preferably a vaccine, according to the present invention.

Further preferred is a method for the treatment and/or prophylaxis as described herein, wherein said method further comprises the administration of an active agent selected from additional antibiotics and/or preparations improving the activity of the immune system of the individual.

Another aspect of the present invention then relates to a method for screening for an antibacterial substance, comprising the steps of a) mixing an isolated peptide according to the present invention (i.e. a protein comprising an Orf13 sequence according to SEQ ID No. 1, a homolog of Orf13 being to at least 80% identical with said protein on the amino acid level, an immunogenic peptide derived from said protein according to SEQ ID No. 1 or said homolog) with a sample suspected of containing an antibacterial substance, and b) monitoring said mixture for binding between said isolated peptide and an antibacterial candidate substance.

One preferred strategy in order to isolate and synthesize antibacterial substances is to label (e.g. with a fluorescent label, an enzymatic label, a mass label, or the like) an isolated peptide according to the present invention, and to measure changes in the label upon interaction (e.g. binding) of an antibacterial candidate substance to said peptide. Another alternative is a phage display assay, which could identify ORF13 binding peptides that could be used to, e.g., inhibit the channel forming activity of ORF13.

Therefore, preferred is a method for screening for an antibacterial substance according to the present invention which further comprises the step of determining the effect of said antibacterial candidate substance on the formation of channels by Orf13 or a homologue thereof as described above.

Yet another aspect of the present invention then relates to a diagnostic assay for detecting the presence of an Orf13 peptide (antigen) or a homolog (antigen) thereof according to the present invention in a sample, comprising the steps of: a) mixing an antibody or fragment thereof according to the present invention with a sample suspected of containing an Orf13; and b) monitoring said mixture for binding between said Orf13 antigen and said antibody or fragment thereof in said sample.

Preferred is a diagnostic assay according to the present invention, wherein said antibody or fragment thereof is immobilized on a solid matrix. Both or either of the antibody or fragment thereof or the Orf13 peptide (antigen) or a homolog (antigen) thereof can be labeled (e.g. with a fluorescent label, an enzymatic label, a mass label, or the like) for the diagnosis.

Multiresistant gram-positive bacteria are a continuing and rising threat in hospitals world-wide. Complex mechanisms enable these pathogens to exchange genetic information and especially the distribution of resistance determinants leads to their propensity to cause hospital outbreaks as well as endemic spread. As presented in the present invention, one of the very factors responsible for genetic exchange, a specific protein of the so-called type 4 secretion systems, represents an Achilles heel that now can be harnessed to fight these often untreatable infections.

A prototypical gram-positive type 4 secretion system is present on plasmid pIP501 and several proteins of the transfer region have been expressed in E. coli. Rabbit sera were raised against two proteins, i.e. ORF10 and ORF13, that code for an ATPase and a putative channel component. The rabbit sera were used in an opsonophagocytic assay and sera raised against ORF13 showed a killing of 99.2% at a dilution of 1:10 against the homologous strain. Using absorption of the sera with increasing amounts of purified protein this killing could be inhibited by up to 44.5%. Testing of a larger collection of strains from different species revealed that 2/2 (100%) E. faecalis, 2/4 (50%) E. faecium, and 5/5 (100%) S. aureus were effectively killed by anti-ORF13 at a dilution of 1:10, including one vancomycin-resistant S. aureus strain and USA300 CA-MRSA. Western Blot analysis demonstrated cross-reactive protein bands in 2 of 3 tested E. faecalis-, 3 of 4 E. faecium- and in all 5 S. aureus strains, respectively, thus indicating a similar situation in said strains. The homologous strain E. faecalis JH2-2pIP501 (expressing protein Orf13) showed a band at 37.5 kDa. In contrast to the control strain, all S. aureus strains showed a band at 50 kDa and the other Enterococcus strains showed a band of 55 kDa. Using a mouse bacteremia model, significant reductions in colony counts were seen in animals that had received anti-ORF13 as compared to animals that had received antisera against ORF 10 and were challenged with the homologous E. feacalis as well as with an E. faecium and a CA-MRSA strain. These data demonstrate that ORF13 is the target of opsonic and protective antigens and therefore is a promising and broadly cross-protective vaccine candidate targeting multi-resistant gram-positive pathogens.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

In the accompanying figure and the accompanying sequence listing,

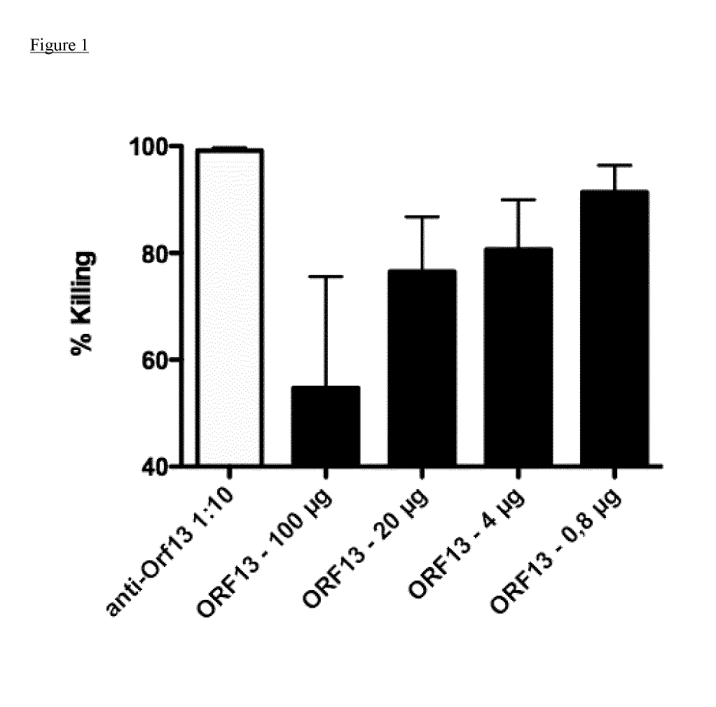

FIG. 1 shows the result of the opsonophagocytic assay according to the Example below.

White bar: unabsorbed serum raised against ORF13; black bars: anti-ORF13 serum absorbed with purified ORF13 protein.

SEQ ID No. 1: shows the amino acid sequence of Orf13 as isolated from the Enterococcus faecalis plasmid pIP501 according to the GenBank Accession number AJ505823.1.

### EXAMPLE

Materials and Methods:

Orf13 consists of 322 amino acids (molecular weight of 37479.76 Da) with a pI of 8.86. orf13 was cloned into an E. coli expression vector with amino-terminal 7×His-tag (Abajy et al., 2007), over-expressed and purified via affinity chromatography (Gossweiner-Mohr, N., Grohmann, E., and Keller, W., unpublished data). The purified protein was used to immunize a rabbit; similar prepared sera against ORF7 and ORF10 were used as controls.

Hyperimmune rabbit sera were examined by opsonophagocytic assay using leukocytes prepared from human volunteers and baby rabbit serum as complement source (Theilacker et al. Mol Microbiol 2009). Using undiluted serum there was 37.7% killing using the anti-ORF7 serum and no killing using anti-ORF8 and anti-ORF11 serum. Serum against ORF13, however, led to a 82.9% killing. Using E. faecalis type 9 as a control (since this strain is known to contain no T4SS) only 14% killing was observed using anti-ORF13 serum.

To confirm specificity a serum dilution of 1:10 was used and varying amounts of purified antigen were used as specific inhibitors. Preabsorption of the anti-ORF13 serum with 100 ug of purified protein led to a 45% inhibition, while lower amounts of inhibitory protein resulted in lower inhibition of killing (see FIG. 1). These data confirm that ORF13 is the target of opsonic antibodies, and that absorption with this purified protein is able to remove the killing ability of the hyperimmune serum raised against ORF13.

In addition, Orf13 has homologs in two E. faecium strains E1679 (catheter tip isolate) and on the E. faecium plasmid pVEF3 (protein p51), on five E. faecalis strains: HIP11704 (unnamed plasmid), E. faecalis DS5 (unnamed plasmid), E. faecalis RE25 (plasmid pRE25, T4SS almost identical with pIP501), E. faecalis DS5 (plasmid pAMbetal) and in one S. pyogenes strain (plasmid pSM19035) (see also above). Therefore, ORF13 seems to be present in several E. faecalis and E. faecium strains and is a promising vaccine target for active or passive immunotherapy of pathogenic bacteria, such as, for example, enterococci.

### LITERATURE AS CITED

1. Abajy, M. Y. Molekularbiologische and biochemische Untersuchungen zum Typ IV Sekretion-ähnlichen System (T4SLS) des konjugativen Antibiotikaresistenzplasmids pIP501 in Enterococcus faecalis. (2007). PhD Thesis, T U Berlin, Berlin.

2. Abajy, M. Y., Kopec, J., Schiwon, K., Burzynski, M., Doring, M., Bohn, C., and Grohmann, E. (2007). A type IV-secretion-like system (T4SLS) is required for conjugative DNA transport of plasmid pIP501 with broad host range in Gram-positive bacteria J. Bacteriol. 189: 2487-2496.

3. Alvarez-Martinez, C. E., and Christie, P. J. (2009). Biological diversity of prokaryotic type IV secretion systems. Microbiol. Mol. Biol. Rev. 73: 775-808.

4. Grohmann, E., Muth, G., and Espinosa, M. (2003). Conjugative plasmid transfer in Gram-positive bacteria. Microbiol. Mol. Biol. Rev. 67: 277-301.

5. Grohmann, E. (2006). Mating cell-cell channels in conjugating bacteria. In Cell-Cell Channels (eds. Baluska, F., Volkmann, D., and Barlow, P. W.), Landes Biosciences, Georgetown, Tex., pp. 21-38. ISBN: 1-58706-065-5.

6. Kurenbach, B., Bohn, C., Prabhu, J., Abudukerim, M., Szewzyk, U., and Grohmann, E. (2003). Intergeneric transfer of the Enterococcus faecalis plasmid pIP501 to Escherichia coli and Streptomyces lividans and sequence analysis of its tra region. Plasmid 50: 86-93.

7. Schaberg, D. R., Clewell, D. B., and Glatzer, L. 1982. Conjugative transfer of R-plasmids from Streptococcus faecalis to Staphylococcus aureus. Antimicrob. Agents Chemother. 22:204-207.

8. Zhu, W., Murray, P. R., Huskins, W. C., Jernigan, J. A., McDonald, L. C., Clark, N. C., Anderson, K. F., McDougal, L. K., Hageman, J. C., Olsen-Rasmussen, M., Frace, M., Alangaden, G. J., Chenoweth, C., Zervos, M. J., Robinson-Dunn, B., Schreckenberger, P. C., Reller, L. B., Rudrik, J. T. and Patel, J. B. Dissemination of an Enterococcus Inc18-Like vanA Plasmid Associated with Vancomycin-Resistant Staphylococcus aureus, Antimicrob. Agents Chemother. 54 (10), 4314-4320 (2010).

9. Theilacker C, Sanchez-Carballo P, Toma I, Fabretti F, Sava I, Kropec A, Holst O, Huebner J. Glycolipids are involved in biofilm accumulation and prolonged bacteraemia in Enterococcus faecalis. Mol Microbiol. 2009 February; 71(4):1055-69.

1. An isolated peptide selected from
a) a protein comprising an Orf13 sequence according to SEQ ID No. 1,
b) a homolog of Orf13 that is at least 80% identical with said protein on the amino acid level,
c) an immunogenic peptide derived from said protein according to a) or b), and
d) an antibody or antigenic fragment thereof that specifically binds to any of a) to c).

2. The peptide according to claim 1, wherein said peptide is a channel-forming component.

3. The peptide according to claim 1, wherein said antibody or antigenic fragment thereof is selected from a monoclonal antibody, an scFV fragment and a Fab-fragment.

4. The peptide according to claim 1, wherein said homolog of Orf13 is encoded by a plasmid isolated from an antibiotic resistant strain of E. faecium, E. faecalis, S. aureus, coagulase-negative staphylococci, Listeria and Clostridia.

5. A pharmaceutical composition comprising the peptide according to claim 1 and a pharmaceutically acceptable carrier, adjuvant and/or diluent.

6. The pharmaceutical composition according to claim 5, wherein said composition is a vaccine.

7. The pharmaceutical composition according to claim 5, further comprising at least one cytokine.

8. The pharmaceutical composition according to claim 6, wherein said vaccine is formulated for administration via intramuscular, subcutaneous, or inhalation routes.

9. A method for the prevention and/or treatment of a disease or condition caused by a bacterium, wherein said method comprises administering, to a subject in need of such prevention or treatment, a peptide of claim 1.

10. The method according to claim 9, wherein the bacterium is selected from enterococci, staphylococci and streptococci.

11. A diagnostic assay for detecting the presence of an Orf13 antigen in a sample, comprising the steps of:
a) mixing an antibody or fragment thereof according to claim 1 with a sample suspected of containing an Orf13; and
b) monitoring said mixture for binding between said Orf13 antigen and said antibody or fragment thereof in said sample.

12. The diagnostic assay according to claim 11, wherein said antibody or fragment thereof is immobilized on a solid matrix.

13. A method for screening for an antibacterial substance, comprising the steps of
a) mixing an isolated peptide according to claim 1 with a sample suspected of containing an antibacterial substance, and
b) monitoring said mixture for binding between said isolated peptide and an antibacterial candidate substance.

14. The method for screening for an antibacterial substance according to claim 13, further comprising the step of determining the effect of said antibacterial candidate substance on the formation of channels by Orf13.

15. The method, according to claim 9, wherein said disease or condition is selected from bacterial infection, enterococcal infection, urinary tract infections, bacteremia, bacterial endocarditis, peritonitis, wound and soft tissue infections, meningitis, and pneumonia.

16. The method, according to claim 10, wherein the bacterium is selected from E. faecium, E. faecalis, S. aureus, coagulase-negative staphylococci and S. pyogenes.

17. The method, according to claim 16, wherein the bacterium is an antibiotic-resistant strain.
